(19) United States
(12) Patent Application Publication
(10) Pub. No.: US 2022/0088359 A1
(43) Pub. Date: Mar. 24, 2022

(54) ELECTROCHEMICAL GASOTRANSMITTER GENERATING COMPOSITIONS AND BIMETALLIC CELLS FOR THE GENERATION OF GASOTRANSMITTERS

(71) Applicant: Noxsano Inc., Columbus, OH (US)

(72) Inventors: Alan Willey, Cincinnati, OH (US); Stevan Samuel, Cincinnati, OH (US); Jacob Robert Adams, Cincinnati, OH (US)

(21) Appl. No.: 17/422,380

(22) PCT Filed: Jan. 13, 2020
(86) PCT No.: PCT/US2020/013291

§ 371 Date: Jul. 12, 2021

Related U.S. Application Data
(60) Provisional application No. 62/791,317, filed on Jan. 11, 2019.

(51) Int. Cl. A61M 35/00; A61K 33/04; A01N 59/02; A01N 59/00; C01B 21/50; C01D 5/14

(52) U.S. Cl. CPC A61M 35/30; A61K 33/04; A01N 59/02; A61M 2202/02; C01B 21/50; C01D 5/14; A61M 2202/0275; A01N 59/00

(57) ABSTRACT

Therapeutic dressings, a process for the preparation of a gasotransmitter, and various methods are disclosed. A therapeutic dressing according to an embodiment includes a composition including an organic electrochemical mediator configured to reduce a gasotransmitter salt, and the gasotransmitter salt converting into a gasotransmitter upon reduction; a carrier adapted to contain the composition; and a bimetallic cell delivering current to the composition.

### FIELD

The present disclosure relates to compositions that include one or more organic electrochemical mediators to generate one or more gasotransmitters, especially nitric oxide and hydrogen sulfide, by utilizing a galvanic cell. The present disclosure also relates to bimetallic cells for generating gasotransmitters from the claimed compositions. The present disclosure also relates to therapeutic dressings, methods, and systems incorporating a galvanic cell.

### BACKGROUND

In the last two decades the importance of gasotransmitters in biological processes has been recognized, especially that of nitric oxide (NO) and hydrogen sulfide (H₂S) which have been implicated in a number of bioregulatory processes including normal physiological control of blood pressure, macrophage destruction of foreign pathogens, ischemic reperfusion injury, hemorrhagic shock, platelet aggregation, and neurotransmission. Recent research has further demonstrated that gasotransmitters possess a broad spectrum of antimicrobial and antiviral activity and may be used as an alternative to conventional antibiotics for drug resistant bacteria. In addition, gasotransmitters may also be used to alleviate inflammation and promote wound healing. However, as gases at ambient temperature and atmospheric pressure, and with short half-lives in physiological milieu, it has proven relatively challenging to deliver gasotransmitters in a controlled and targeted manner.

One attempt to deliver nitric oxide in a controlled and targeted manner is to modify a polymer such that it decomposes when moist or at elevated temperature to release nitric oxide, an approach described by BASF in US application U.S. Ser. No. 13/975,930 and Novan Inc. in US 20140134321 and US 20140171395. While these polymers allow nitric oxide to be delivered from a solid there is limited control over the release profile.

Another attempt to deliver nitric oxide in a controlled and targeted manner is through acid catalyzed decomposition of nitrite salts as described by Edixomed in WO/2014/188175A. This approach also delivers nitric oxide from a solid formulation but with limited control over the release profile.

A further attempt to deliver nitric oxide in a controlled and targeted manner is through electrochemical reduction of nitrite to nitric oxide as described in U.S. Pat. No. 9,480,785. The electrochemical generation of nitric oxide is achieved using a copper catalyst and allows for control over the release profile. This approach, like the embodiments described herein, utilizes electrochemical reduction of nitrite to nitric oxide. In contrast to U.S. Pat. No. 9,480,785 the present embodiments use an organic mediator compatible with open wounds unlike the metal catalyst.

The application of nitric oxide has been relatively limited because of the absence of a controlled and targeted delivery method or material. Therefore, there is a need for a gasotransmitter delivery system, that can deliver these species in a temporally, spatially and targeted manner.

### SUMMARY

One embodiment is directed to a therapeutic dressing including a composition including an organic electrochemical mediator configured to reduce a gasotransmitter salt, and the gasotransmitter salt converting into a gasotransmitter upon reduction; a carrier adapted to contain the composition; and a bimetallic cell delivering current to the composition. Other embodiments are directed to bimetallic cells, processes for the preparation of a gasotransmitter, methods of treatment using a gasotransmitter, and other methods using a gasotransmitter.

This summary is not intended to identify key or essential features of the claimed subject matter, nor is it intended to be used as an aid in limiting the scope of the claimed subject matter. Further embodiments, forms, features, and aspects of the present application shall become apparent from the description and figures provided herewith.

### DETAILED DESCRIPTION

The foregoing and other aspects of the present disclosure will now be described in more detail with respect to the description and methodologies provided herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

U.S. Pat. No. 10,342,706 to Willey et al. may include subject matter that is relevant to the present disclosure, and the subject matter of such patent is hereby incorporated by reference in its entirety

The terminology used in the present description is for the purpose of describing some embodiments only and is not intended to be limiting of the invention. The term gasotransmitter salt refers to salts that when used in the present embodiments generate gasotransmitters. As used in the description of the embodiments and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this present disclosure belongs. As used herein, the terms “bimetallic” and “galvanic” are interchangeable.

The present embodiments relate to electrochemically mediated compositions compromising an electrochemical mediator and a nitrite, nitrate, sulfite, thiosulfate, thiosulfate or sulfate salt. Still further, the present embodiments also relate to methods for providing one or more health benefits to a subject by exposing a targeted site to the gasotransmitter generating composition. In some embodiments, the amount and rate of the release of gasotransmitters is regulated by adjusting the formulation and/or voltage applied by the bimetallic cell device.

When two metals or semi-metals are submerged in an electrolyte, they experience galvanic corrosion. The rate of corrosion is determined by the electrolyte, position on the galvanic (electrochemical) series, and the relative areas of the anode and cathode exposed to the electrolyte. The difference can be measured as a difference in voltage potential: a less noble metal is the one that has a lower (that is, more negative) electrode potential than the nobler one, and will function as the anode (electron or anion attractor) within the electrolyte device functioning as described above (a galvanic cell). Galvanic reaction is the principle upon which batteries are based. In common usage, the word “battery” has come to include a single galvanic cell, but a battery properly consists of multiple voltaic cells. Each cell consists of two half-cells connected in series by a conductive electrolyte containing metal cations. One half-cell includes electrolyte and the negative electrode, the electrode to which anions (negatively charged ions) migrate; the other half-cell includes electrolyte and the positive electrode, to which cations (positively charged ions) migrate. Connecting multiple cells together in a battery provides enough, stable current for electrical devices or electrolysis. A galvanic cell based on two dissimilar materials (from the galvanic series) in an electrolyte generally provides insufficient stable current for application. It has surprisingly been found that a galvanic cell does provide enough current for the generation of gasotransmitters as described herein. When two members of the galvanic series are immersed in a gasotransmitter salt solution of the present disclosure a potential is created and current flows. This current leads to generation of the gasotransmitter from the gasotransmitter salt. Additionally, it has been found that by selecting materials from the galvanic series with different potential gaps and varying the electrolyte concentration the rate of production of the gasotransmitter can be controlled.

The present disclosure also provides treatment systems, bandages, and dressings comprising an organic electrochemical mediator, and a gasotransmitter generating salt which upon reduction, generates a gasotransmitter (especially nitric oxide or hydrogen sulfide), and an anode and cathode selected from a galvanic series, the galvanic series selected so as to provide the required reduction potential required to reduce the gasotransmitter generating salt via the organic electrochemical mediator. The present disclosure thus includes reduction systems to be used to reduce, directly or indirectly, gasotransmitter systems, including those that may be advantageously and conveniently applied and incorporated into dressing and bandage systems, including those adapted to vary the duration of the electric connection governing the galvanic reaction, or otherwise suspend or terminate the galvanic reaction, such as exemplified by those described herein.

In some embodiments, the amount and rate of the release of gasotransmitter is regulated by adjusting the formulation of the one or more galvanic cell components in order to vary the gasotransmitter-generating current brought to bear on the organic electrochemical mediator and thereby to the gasotransmitter generating salt.

It will be appreciated that the relative concentration of the electron donor, gasotransmitter salt and source of galvanic reducing current may be determined for any given photocatalyzable composition depending upon the desired gasotransmitter production and duration of that production, considering the amount and duration of current to which the gasotransmitter-generating composition is to be exposed over time in accordance with the desired gasotransmitter production and duration of that production.

The galvanic action of the galvanic cell, and thereby the resultant gasotransmitter-generation, may be varied through any one or more techniques or methods known in the art with regards the electrochemical principles applicable to such arrangements, such as by varying the amounts, concentration and/or physical disposition of the galvanic cell with respect to the gasotransmitter-generating composition. Accordingly, the systems, dressings, bandages of the present disclosure may incorporate optional arrangements so as to be able to vary the gasotransmitter-generating current within a given system, dressing, or bandage. Examples of such arrangements may be in the form of the use of a galvanic cell comprising a series of individual cell pairs of relatively equal galvanic strength that are brought into electrical contact with the gasotransmitter-generating composition in series over time, such that the voltage of the galvanic cell is refreshed over time. As another example, in the case where an increase of resultant gasotransmitter-generation over time is desired, a series of individual cell pairs of increasing galvanic strength are brought into electrical contact with the gasotransmitter-generating composition in series over time, so as to increase the rate of gasotransmitter-generation over time. This may be done by providing individual compartment layers within a bandage, dressing or other treatment system that are provided with releasable layers so as to be adapted to be brought into electrical contact with the gasotransmitter-generating composition in series over time. In another embodiment, the treatment system, such as a bandage or dressing may be provided in a kit or otherwise with two or more containers or layerable bandage or dressing components, such as may be placed in electrical contact with the gasotransmitter-generating composition held by the bandage or dressing and subsequently removed in favor of a subsequent one of such layerable bandage or dressing components, so as to provide successive amounts of gasotransmitter-generating current over a desired treatment schedule over which varied gasotransmitter-generation is desired.

### Electrochemical Mediator

The electrochemical mediator consists an organic redox moiety and a solubilizing moiety. In one embodiment the redox moiety is an organic moiety that is reduced at an electrode at a potential of from about −0.1 V to about −2.0 V in some embodiments, from about −0.5 V to about −1.7 V in some embodiments, and from about −0.75 V to about −1.5 V in some embodiments. Upon reduction the redox mediator forms a single electron reduced species that diffuses in the aqueous solution and reduces the gasotransmitter generating salt via electron transfer. In order to reduce the gasotransmitter generating salt the mediator should have a reduction potential, in its reduced state, more negative than that required for the reduction of the salt to the gasotransmitter.

The redox moiety of the electrochemical mediator of the present embodiments is selected from the group consisting of water soluble ketones, benzophenones, quinones, fluoresceins, xanthones, thioxanthones.

The electrochemical mediators of the present embodiments may incorporate polar functional groups, such as the alcohol, amine, amide, carboxylic acid, sulfonic acid, and phosphate groups, which either ionize or are capable of relatively strong intermolecular forces of attraction with water (hydrogen bonding). In some embodiments, acidic and basic groups are useful. As used herein, the term “water solubilizing moiety” refers to a moiety that is attracted to water and dissolves in water to form a homogenous solution. In one embodiment, the water solubilizing moiety is selected from the group consisting of alcohol, amine, amide, carboxylic acid, sulfonic acid and phosphate groups. In another embodiment, the hydrophilic moiety is selected from the group consisting of water soluble oligomers, water soluble polymers and water soluble copolymers. In one embodiment, the hydrophilic moiety may be selected from carboxylic acid and sulfonic acid. In another embodiment, the hydrophilic group is selected from the group consisting of alkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, arginine, histidine, aspartic acid, glutamic acid, serine, threonine, asparagine, glutamine, isopropyl acrylamide, styrene sulfonic acid, vinyl methyl ether, vinylphosphonic acid, ethylene imine, and mixtures thereof. In one embodiment, the hydrophilic moiety may be selected from the group consisting of alkylene oxide oligomer polymers, alkylene oxide oligomer copolymers, vinyl alcohol, vinyl pyrrolidone, acrylic acid, acrylamide, cellulose, and mixtures thereof.

As used herein, the term “redox moiety” refers to an organic moiety that is capable of being reduced at an electrode to form a radical anion that in turn will reduce the gasotransmitter salt.

### Electrochemical Composition

The present embodiments relate to an electrochemical composition that includes the electrochemical mediator, as described in further detail above, and a gasotransmitter salt.

The electrochemical composition may be an aqueous solution, emulsion, solid, gel, hydrogel, or incorporated into a material, such as a film.

In one embodiment, the electrochemical composition comprises the electrochemical mediator, gasotransmitter salt, water and a super absorbent hydrogel. The gel composition thus formed may be applied directly to a wound along with a bimetallic dressing or applied to a wound with the composition contained in a pouch dressing. It will be understood that the bimetallic cell may be incorporated into the pouch dressing or separate and applied to the pouch dressing.

In another embodiment, the individual components of the electrochemical composition may be incorporated into both a composition and a material, such as a film. In one embodiment, the electrochemical mediator may be included in a film and the nitrite may be included in a composition. It will be understood that in this embodiment, a film comprising an electrochemical mediator may be applied to surface and a composition comprising a gasotransmitter salt may be applied separately.

However, if the electrochemical mediator is in aqueous composition, the composition may comprise from 0.1% to 99%, by weight of the composition, of water. It will therefore be understood that the electrochemical mediator can be in concentrated or diluted form. It is further contemplated that a portion of the water may be replaced with another solvent such as ethanol, glycol, glycol-ethers, glycerin, water soluble acetates, ethers, and alcohols.

As noted above, the present embodiments relate to electrochemical compositions that include the electrochemical mediator and a gasotransmitter salt. In such embodiments, it will be understood that the electrochemical mediator is reduced at an electrode then reacts in the reduced form with gasotransmitter salt to generate the gasotransmitters. It will also be understood that the gasotransmitter salt can be converted into a gasotransmitter upon reaction with the electrochemical mediator after electron transfer at the electrode. It will be understood that the electrochemical mediator is substantially unreactive with the gasotransmitter salt without activation by the electrode and that the gasotransmitter salt is not substantially reduced directly by the electrode.

In the present embodiments, electron transfer to the electrochemical mediator at the electrode allows the reaction to progress to create gasotransmitters. In some embodiments, the gasotransmitter may act to control blood pressure, macrophage destruction of foreign pathogens, and neurotransmission, provide a broad-spectrum of antimicrobial activity, and alleviate inflammation, and promote wound healing.

### Gasotransmitter Salt

The electrochemical composition of the present embodiments comprises a gasotransmitter salt. When used in the electrochemical composition of the present embodiments, the gasotransmitter salt is converted into a gasotransmitter by reduction.

In some embodiments, the gasotransmitter salt is a nitrite or nitrate salt with the formula:

A[NOₓ]ₘ
wherein x is 2 or 3 and A is selected from the group consisting of monovalent cations, divalent cations, and trivalent cations.

In some embodiments, A is selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, aryl-ammonium, and mixtures thereof. In some embodiments, A is selected from the group consisting of lithium, sodium, potassium, magnesium, calcium, ammonium, and mixtures thereof m denotes the number of anions required to balance the charge of the cation yielding a neutral nitrite or nitrate salt.

In other embodiments, the gasotransmitter salt is a sulfate, sulfite, or thiosulfate salt with the formula:

A[SxOy]m
wherein A is selected from the group consisting of monovalent cations, divalent cations, and trivalent cations.

In some embodiments, A is selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, aryl-ammonium, and mixtures thereof. In some embodiments, A is selected from the group consisting of lithium, sodium, potassium, magnesium, calcium, ammonium, and mixtures thereof x is 1 or 2, y is 3 or 4 and m denotes the number of anions required to balance the charge of the cation yielding a neutral sulfate, sulfite, or thiosulfate salt.

### Bimetallic Cell Device

In order to generate gasotransmitters from the compositions described herein, a voltage must be applied to the composition. The voltage applied must be greater than the reduction potential of the electrochemical mediator in the gasotransmitter generating composition. Though the form of the device may be tailored to the desired application, the device may contain the following elements:
Bimetallic electrodes (i.e., two electrodes formed from different metals) that contact the composition and transfer electrons to and from said composition. The electrodes are selected from two dissimilar materials of the galvanic series. In some embodiments, the electrode is flexible.
A non-conductive substrate including but not limited to polyethylene, polypropylene and polyester films and non-wovens, cellulose substrates such as cotton, chitosan, and collagen.

Those skilled in the art will understand the galvanic series is divided into cathodic materials and anodic materials relative to a standard electrode such as the saturated calomel electrode (SCE). If two materials are chosen with different potentials from the galvanic series and placed in a conducting salt solution, a potential is generated between the two materials. The bimetallic cell device comprises electrodes selected from the galvanic series such that the potential difference between the two materials is greater than the reduction potential of the mediator. Anodic materials suitable for the present embodiments include but are not limited to magnesium zinc, beryllium, aluminum, tin, and copper. Cathodic materials suitable for the present embodiments include but are not limited to carbon, gold, silver, platinum, and nickel. As will be appreciated, the following materials may be incorporated into the bimetallic cell devices of the present disclosure: magnesium, zinc, beryllium, aluminum alloys, cadmium, mild steel, cast iron, low alloy steel, austenitic cast iron, aluminum bronze, naval brass, yellow brass, red brass, tin, copper, 50/50 lead tin solder, admiralty brass, aluminum brass, manganese bronze, silicon bronze, stainless steel, nickel silver, 90/10 copper nickel, 80/20 coper nickel, stainless steel, 70/30 copper nickel, nickel aluminum bronze, nickel chromium alloy, nickel, silver, nickel iron chromium alloy, titanium, gold, platinum, and graphite.

It will be understood that the galvanic series may be selected to provide sufficient reductive potential to reduce the “redox moiety” at an electrode to form a radical anion that in turn may reduce the gasotransmitter salt.

### Optional Additives

The electrochemical compositions of the present embodiments may also contain additional adjunct additives. The precise nature of these additional components and levels of incorporation thereof will depend on the physical form of the composition, and the precise nature of the cleaning, disinfecting, or health benefit for which it is to be used. It will be understood that some of the adjunct additives noted below will have electrochemical properties, but it will be further understood that such additives will not replace the components noted above.

### Topical Compositions

According to some embodiments, provided herein are topical compositions. In some embodiments a topical composition is in the form of a hydrogel. “Hydrogel,” as used herein, refers to a hydrophilic gel comprising a gel matrix and water. In some embodiments, a topical composition comprises at least one polyhydric alcohol, at least one viscosity increasing agent, and water.

Exemplary polyhydric alcohols that may be present in a composition of the present embodiments include, but are not limited to, glycerol, propylene glycol, polyethylene glycol, polypropylene glycol, triethylene glycol, neopentyl glycols, triethanolamine, diethanolamine, ethanolamine, butylene glycol, polyethylene glycol, n-methyl diethanolamine, isopropanolamine, sorbitol, arabitol, erythritol, HSH, isomalt, lactitol maltitol, mannitol, xylitol, threitol, ribitol, galactitol, fucitol, iditol, inositol, volemitol, and any combination thereof. In some embodiments, a composition of the present disclosure comprises glycerol.

A polyhydric alcohol may be present in a composition of the present embodiments in an amount of about 1% to about 30% by weight of the composition or any range and/or individual value therein, such as, but not limited to, about 1% to about 20% or about 5% to about 15% by weight of the composition. In certain embodiments, a polyhydric alcohol is present in a composition of the present disclosure in an amount of about 1%, 2%, 3%, 4%, 5%, 6%, 7%, 8%, 9%, 10%, 11%, 12%, 13%, 14%, 15%, 16%, 17%, 18%, 19%, or 20% by weight of the composition or any range and/or individual value therein.

Exemplary viscosity increasing agents that may be present in a composition of the present embodiments include, but are not limited to, a carboxypolymethylene; a polyacrylic polymer such as polyacrylic acid, a polyacrylate polymer, a cross-linked polyacrylate polymer, a cross-linked polyacrylic acid, and mixtures thereof; a cellulose ether such as hydroxyalkyl cellulose polymers such as hydroxypropyl methyl cellulose (HPMC), hydroxypropyl cellulose, hydroxyethyl cellulose, methyl cellulose, carboxymethyl cellulose, and mixtures thereof; a methacrylate; a polyvinylpyrrolidone; cross-linked polyvinyl pyrrolidone; polyvinylpyrrolidone-vinyl acetate copolymer; polyvinyl alcohol; polyethylene oxide; polyethylene glycol; polyvinyl alkyl ether-maleic acid copolymer; a carboxy vinyl polymer; a polysaccharide; a gum such as sodium alginate, carrageenan, xanthan gum, gum acacia, arabic gum, guar gum, pullulan, agar, chitin, chitosan, pectin, karaya gum, zein, hordein, gliadin, locust bean gum, tragacantha, and mixtures thereof; a protein such as collagen, whey protein isolate, casein, milk protein, soy protein, gelatin, and mixtures thereof; a starch such as maltodextrin, amylose, high amylose starch, corn starch, potato starch, rice starch, tapioca starch, pea starch, sweet potato starch, barley starch, wheat starch, waxy corn starch, modified starch (e.g. hydroxypropylated high amylose starch), dextrin, levan, elsinan, gluten, and mixtures thereof; bentonite; calcium stearate; ceratonia; colloidal silicon dioxide; dextrin; hypromellose; polycarbophil; kaolin; saponite; sorbitan esters; sucrose; sesame oil; tragacanth; potassium alginate; povidone; sodium starch glycolate; phospholipids; and any combination thereof.

In some embodiments, the composition comprises a carboxypolymethylene, such as, but not limited to, those commercially available from Lubrizol Corporation of Wickliffe, Ohio under the trade name Carbopol®. Exemplary Carbopol® polymers that may be present in a composition of the present embodiments include, but are not limited to, Carbopol® 974P NF polymer, such as Type A, Type B and/or Type C Homopolymers; Carbopol® Ultrez 10, 20, 21 NF polymer; Carbopol® 971P NF polymer; Carpobol® 980P polymer, Carbopol® ETD 2020 NF polymer, Carbopol® 71G NF polymer, Carbopol® 981P NF polymer, Carbopol® 970P NF polymer, Carbopol® 981 P NF polymer, Carbopol® 5984P NF polymer, Carbopol® 934P NF polymer, Carbopol® 940P NF polymer, Carbopol® 941P NF polymer, Carbopol® 13242 NF polymer, Carbopol® AA-1 USP NF polymer, Carbopol® TR1 NF polymer, Carbopol® TR2 NF polymer, Lubrizol Aqua CC polymer and SF-2 polymer, and any combination thereof.

A viscosity increasing agent may be present in a composition of the present embodiments. In some embodiments, the composition comprises at least two viscosity increasing agents that may be the same or different. In some embodiments, a first viscosity increasing agent may be present in a composition of the present disclosure in an amount of about 0.01% to about 5% by weight of the composition or any range and/or individual value therein, such as, but not limited to, about 0.05% to about 3% or about 0.1% to about 1.5% by weight of the composition. In certain embodiments, a first viscosity increasing agent is present in a composition of the present disclosure in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5% by weight of the composition or any range and/or individual value therein.

Water may be present in a composition of the present embodiments in an amount of about 0.1% to about 99% by weight of the composition or any range and/or individual value therein, such as, but not limited to, about 75% to about 95% or about 80% to about 90% by weight of the composition. In certain embodiments, water is present in a composition of the present disclosure in an amount of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% by weight of the composition or any range and/or individual value therein.

In some embodiments, the composition comprises at least one polyhydric alcohol present in an amount of about 1% to about 30% by weight of the composition, at least one viscosity increasing agent present in an amount of about 0.1% to about 5% by weight of the composition, and water present in an amount of about 70% to about 99% by weight of the composition. The composition may be in the form of a hydrogel. In certain embodiments, the viscosity increasing agent may be a carboxypolymethylene.

A composition of the present embodiments may comprise a preservative. A preservative may be present in a composition of the present embodiments in an amount of about 0.01% to about 1% by weight of the composition or any range and/or individual value therein, such as, but not limited to, about 0.05% to about 1% or about 0.1% to about 1% by weight of the composition. In certain embodiments, a preservative is present in a composition of the present disclosure in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% by weight of the composition or any range and/or individual value therein. Exemplary preservatives that may be present in a composition of the present embodiments include, but are not limited to, sorbic acid, benzoic acid, methylparaben, propylparaben, methylchloroisothiazolinone, methylisothiazolinone, diazolidinyl urea, chlorobutanol, triclosan, benzethonium chloride, p-hydroxybenzoate, chlorhexidine, digluconate, hexadecyltrimethylammonium bromide, alcohols, benzalkonium chloride, boric acid, bronopol, butylparaben, butylene calcium acetate, calcium chloride, calcium lactate, carbon dioxide, cationic, and bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, sodium metabisulfite, xylitol, sulphur dioxide, carbon dioxide, and any combination thereof.

A composition of the present embodiments may comprise a neutralizing agent. A neutralizing agent may be present in a composition of the present embodiments in an amount sufficient to provide the composition with a pH of about 3 to about 8, or any range and/or individual value therein, such as, but not limited to, about 4 to about 7 or about 6 to about 7. In some embodiments, a neutralizing agent adjusts the pH of the composition. In certain embodiments, a neutralizing agent is present in the composition in an amount sufficient for the composition to have a pH of about 3, 3.5, 4, 4.5, 5, 5.5, 6, 6.5, 7, 7.5, or 8 or any range and/or individual value therein. Exemplary neutralizing agents that may be present in a composition of the present embodiments include, but are not limited to, bases such as sodium hydroxide, potassium hydroxide, and mixtures thereof; acids such as hydrochloric acid, citric acid, acetic acid, and mixtures thereof; sodium carbonate; trolamine; tromethamine; aminomethyl propanol; triisopropanolamine; aminomethyl propanol; tetrahydroxypropyl ethylenediamine; tetrasodium EDTA; suttocide A; and any combination thereof.

A composition of the present embodiments may be unbuffered or buffered. In some embodiments, the composition may be unbuffered. In other embodiments, the composition may be buffered. Exemplary buffers that may be present in the composition of the present disclosure include, but are not limited to, acetic acid/acetate buffers; hydrochloric acid/citrate buffers; citro-phosphate buffers; phosphate buffers; citric acid/citrate buffers; lactic acid buffers; tartaric acid buffers; malic acid buffers; glycine/HCl buffers; saline buffers such as phosphate buffered saline (PBS), Tris-buffered saline (TBS), Tris-HCl, NaCl, Tween buffered saline (TNT), phosphate buffered saline, Triton X-100 (PBT) and mixtures thereof; cacodylate buffers; barbital buffers; tris buffers; and any combination thereof.

In certain embodiments, a composition of the present embodiments may comprise a buffering agent. Exemplary buffering agents include, but are not limited to, citric acid, acetic acid, lactic acid, boric acid, succinic acid, malic acid, and any combination thereof. A buffering agent may be present in a composition of the present embodiments in an amount of about 0.01% to about 2% by weight of the composition or any range and/or individual value therein, such as, but not limited to, about 0.05% to about 1%, about 0.1% to about 0.5%, or about 0.1% to about 2% by weight of the composition. In certain embodiments, a buffering agent is present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% by weight of the composition or any range and/or individual value therein.

In some embodiments, a buffer is present in the composition in an amount sufficient for the composition to have a pH of about 3 to about 8, or any range and/or individual value therein, such as, but not limited to, about 4 to about 7 or about 6 to about 7. In certain embodiments, a buffer is present in the composition in an amount sufficient for the composition to have a pH of about 3, 3.5, 4, 4.5, 5, 5.5, 6, 6.5, 7, 7.5, or 8 or any range and/or individual value therein.

A composition of the present embodiments may be antimicrobial. In some embodiments, the composition comprises a preservative that is present in an amount sufficient to provide antimicrobial activity to the composition. In certain embodiments, the composition comprises at least one polyhydric alcohol present in an amount of about 1% to about 30% by weight of the composition, at least one viscosity increasing agent present in an amount of about 0.1% to about 5% by weight of the composition, water present in an amount of about 70% to about 99% by weight of the composition, and at least one preservative in an amount of about 0.01% to about 1% by weight of the composition. The composition may be buffered to have a pH in a range of about 3 to about 8 or about 6 to about 8.

A composition of the present embodiments may have a viscosity in a range of about 5,000 cP to about 25,000 cP or any range and/or individual value therein, such as, but not limited to, about 5,000 cP to about 20,000 cP or about 7,000 cP to about 15,000 cP. In certain embodiments, the composition may have a viscosity of about 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, or 25,000 cP or any range and/or individual value therein.

A composition of the present embodiments may comprise an active pharmaceutical ingredient (API). Any suitable API or combinations of APIs may be included in a composition of the present embodiments. Examples of APIs include, but are not limited to, antimicrobial agents, anti-acne agents, anti-inflammatory agents, analgesic agents, anesthetic agents, antihistamine agents, antiseptic agents, immunosuppressants, antihemorrhagic agents, vasodilators, wound healing agents, anti-biofilm agents, and any combination thereof. Exemplary APIs include, but are not limited to, those described in International Application Publication No. WO 2013/006608. Alternatively, a composition of the present embodiments may not comprise an API.

### Pharmaceutical Compositions

The present embodiments provide pharmaceutical compositions that may be administered topically. A pharmaceutical composition of the present embodiments may comprise, consist essentially of, or consist of a hydrophobic base and an amphiphilic compound. In some embodiments, a pharmaceutical composition further comprises a moisture activated API. A pharmaceutical composition of the present embodiments may comprise an ointment, salve, cream, and/or the like.

“Hydrophobic base” as used herein refers to a natural and/or synthetic fat, wax, oil, and/or the like. Any suitable hydrophobic base may be used in a pharmaceutical composition of the present embodiments. In certain embodiments, a pharmaceutical composition comprises two or more hydrophobic bases, such as, but not limited to, 2, 3, 4, 5, or more hydrophobic bases. Exemplary hydrophobic bases include, but are not limited to, branched and unbranched hydrocarbons, branched and unbranched hydrocarbon waxes, vaseline, hydrocarbon gel, liquid paraffin, white petrolatum, petrolatum, microcrystalline wax, candelilla wax, carnauba wax, lanolin (wool wax), wool wax alcohol, esparto grass wax, cork wax, guaruma wax, rice bran wax, sugar cane wax, berry wax, ouricury wax, soy wax, jojoba oil, uropygial grease, ceresine, paraffin waxes, micro waxes, plant oils, animal oils, carnauba wax, beeswax, cacao butter, hard fat, mineral oil, vegetable oil, avocado oil, borage oil, canola oil, castor oil, chamomile oil, coconut oil, corn oil, cottonseed oil, rapeseed oil, evening primrose oil, safflower oil, sunflower oil, soybean oil, sweet almond, palm oil, palm kernel oil, arctium lappa seed oil, sesame oil, Borago officinalis seed oil, brassica campestris oleifera oil, brevoortia oil, bubulum oil, cistus ladaniferus oil, Elaeis guineensis oil, almond oil, pine oil, olive oil, peanut oil, wheat germ oil, grape seed oil, thistle oil, lard, tallow, palm olein, illipe butter, shea butter, cocoa butter, kokum butter, sal butter, lecithin, japan wax lanolin, partially hydrogenated vegetable oils, hydrophobic polymers, and any combination thereof.

In some embodiments, a hydrophobic base may comprise a hydrophobic polymer. Any suitable hydrophobic polymer may be used in a pharmaceutical composition of the present embodiments. Exemplary hydrophobic polymers include, but are not limited to hydrocarbon polymers and/or co-polymers, aromatic polyurethanes, silicone rubber, polysiloxanes, polycaprolactone, polycarbonate, polyvinyl chloride, polyethylene, poly-L-lactide, poly-DL-glycolide, polyetheretherketone (PEEK), polyamide, polyimide and polyvinyl acetate. In some embodiments of the present disclosure, a pharmaceutical composition of the present disclosure comprises one or more hydrocarbon polymers and/or co-polymers. In certain embodiments, the pharmaceutical composition comprises one or more hydrocarbon polymers and/or co-polymers, such as, but not limited to, those commercially available from Calumet Specialty Products Partners of Indianapolis, Ind. under the trademark Versagel® and/or those commercially available from Croda International Plc of East Yorkshire, United Kingdom under the trade name Crodabase SQ.

In some embodiments, a hydrophobic polymer may act as thickening and/or gelling agent in a pharmaceutical composition. Specifically, a hydrophobic polymer may act as a visco-elastic substance and may retain the composition at the site of application, along with any compounds dispersed therein (e.g., an API, etc.). A hydrophobic polymer may be present in a pharmaceutical composition of the present disclosure at a concentration from about 30% to about 60% by weight or any range therein, such as, but not limited to, from about 35% to about 55% by weight or about 40% to about 50% by weight.

In some embodiments, a hydrophobic base comprises one or more plant and/or mineral oils. Any suitable oil may be used in the pharmaceutical compositions of the present embodiments. Exemplary mineral oils include, but are not limited to, light mineral oil, white mineral oil, paraffinic oils, naphthenic oils, aromatic oils, and any combination thereof. An oil (e.g., plant and/or mineral oil) may be present in a pharmaceutical composition of the present embodiments at a concentration from about 1% to about 30% by weight or any range therein, such as, but not limited to, from about 5% to about 20% by weight or about 5% to about 15% by weight.

In some embodiments, a hydrophobic base, such as, but not limited to, an oil (e.g., a plant and/or mineral oil), may be used to tune the viscosity and/or spreadability of the pharmaceutical composition. For example, a low viscosity hydrophobic base, such as light mineral, may be used to thin (i.e., reduce the viscosity) a pharmaceutical composition, such as, a pharmaceutical composition comprising a high viscosity hydrophobic base. This may enable the application of a pharmaceutical composition of the present embodiments over a wide area, and may serve to maintain any compounds dispersed therein (e.g., an API, etc.) at the site of application. In certain embodiments, a hydrophobic base comprises a mineral oil and a hydrophobic polymer.

A hydrophobic base may be present in a pharmaceutical composition of the present embodiments at a concentration from about 35% to about 90% by weight or any range therein, such as, but not limited to, from about 40% to about 80% by weight or about 50% to about 70% by weight. In certain embodiments, a hydrophobic base is present in the pharmaceutical composition at a concentration from about 45% to about 55% by weight.

“Amphiphilic compound” as used herein refers to a compound comprising hydrophilic and hydrophobic properties. An amphiphilic compound may comprise two or more compounds, each of which may provide the hydrophilic property and/or the hydrophobic property. In some embodiments, the amphiphilic compound comprises one compound having hydrophilic and hydrophobic properties. In some embodiments, an amphiphilic compound may absorb moisture without substantially absorbing vaporous moisture. The absorption of moisture may allow for activation of a moisture activated API in a pharmaceutical composition of the present embodiments upon contact with the moisture, but not upon contact with vaporous moisture. “Substantially absorbing” (and grammatical variations thereof) as used herein means that the amount of vaporous moisture absorbed is more than 2% by weight of an amphiphilic compound. Thus, an amphiphilic compound of the present embodiments absorbs vaporous moisture by less than about 2%, 1.5%, 1%, 0.5%, 0.25% by weight of an amphiphilic compound or any range therein. In some embodiments, an amphiphilic compound may prevent and/or minimize the pharmaceutical composition from substantially absorbing vaporous moisture, thereby moisture may be present in a pharmaceutical composition of the present disclosure by less than about 2%. In some embodiments, an amphiphilic compound absorbs water vapor by less than about 2% by weight or about 1% by weight. This may minimize and/or prevent a pharmaceutical composition from absorbing water vapor and thus water may be present in a pharmaceutical composition of the present embodiments by less than about 2% by weight or about 1% by weight water. In certain embodiments, an amphiphilic compound absorbs less than about 0.5% by weight water vapor and thus the pharmaceutical composition may comprise less than about 0.5% by weight water.

“Moisture” as used herein refers to a liquid, such as, but not limited to, a bodily fluid such as, but not limited to, blood, sweat, mucus, saliva, sebum, tears, exudate, and/or vaginal secretions; water; deoxygenated water; saline solutions; acidic or alkaline buffer solutions; and/or any combination thereof. “Vaporous moisture” as used herein refers to moisture in the gas phase. For example, vaporous moisture, includes, but is not limited to, water vapor. Thus, in some embodiments, an amphiphilic compound may prevent and/or minimize the absorption of water vapor, thereby, when the API comprises a moisture activated pharmaceutical ingredient, the API in the pharmaceutical composition is not activated by the vaporous moisture (e.g., water vapor). In contrast, an amphiphilic compound may absorb and/or allow moisture (e.g., water, a bodily fluid, etc.) to be absorbed when a pharmaceutical composition of the present embodiments is contacted with the moisture, thereby activating the API when the API comprises a moisture activated API.

An amphiphilic compound may have a hydrophilic-lipophilic balance (HLB) value of 12 to 20 or any range therein, such as, but not limited to, 15 to 20 or 18 to 20. In certain embodiments, an amphiphilic compound comprises a HLB value of 19.

Exemplary amphiphilic compounds include, but are not limited to, fatty acid esters. One or more fatty acid ester(s) may be present in the pharmaceutical compositions of the present embodiments, such as 2, 3, 4, or more fatty acid esters. Exemplary fatty acid esters include, but are not limited to, C6-C22 alkyl and/or alkenyl fatty acid esters such as methyl laurate, ethyl laurate, ethyl myristate, ethyl palmitate, ethyl linoleate, propyl isobutyrate, isopropyl laurate, isopropyl myristate, isopropyl palmitate, oleyl myristate, oleyl stearate, and oleyl oleate; ether-esters such as fatty acid esters of ethoxylated fatty alcohols; polyhydric alcohol esters such as ethylene glycol mono- and di-fatty acid esters, diethylene glycol mono- and di-fatty acid esters; polyethylene glycol (6-2000) fatty acid mono- and/or diesters such as PEG-6-laurate, PEG-6-stearate, PEG-8-dilaurate, PEG-8-distearate, etc.; polyethylene glycol glycerol fatty acid esters such as PEG-20-glyceryl laurate, PEG-20-glyceryl stearate, and PEG-20-glyceryl oleate; propylene glycol mono- and di-fatty acid esters; polypropylene glycol 2000 monooleate; polypropylene glycol 2000 monostearate; ethoxylated propylene glycol monostearate; glyceryl mono- and di-fatty acid esters; polyglycerol fatty acid esters such as polyglyceryl-10 laurate, etc.; ethoxylated glyceryl monostearate; 1,3-butylene glycol monostearate; 1,3-butylene glycol distearate; polyoxyethylene polyol fatty acid ester; sorbitan fatty acid esters including sorbitan trioleate and sorbitan monolaurate; polyethylene glycol sorbitan fatty acid esters such as PEG-6 sorbitan monooleate; polyoxyethylene sorbitan fatty acid esters including polyoxyethylene (20) sorbitan monolaurate; sucrose fatty acid esters such as saccharose monopalmitate and saccharose monostearate; wax esters such as beeswax, spermaceti, myristyl myristate, stearyl stearate and arachidyl behenate; polyethylene glycol alkyl ethers such as PEG-10 oleyl ether or PEG-9 cetyl ether; polyethylene glycol alkyl phenols such as PEG-10-100 nonyl phenol; polyoxyethylene-polyoxypropylene block copolymers such as poloxamer 188; sterol esters such as cholesterol fatty acid esters, and any combination thereof.

A fatty acid ester may comprise a polyethylene glycol (PEG) glyceride. The polyethylene glycol portion of a PEG glyceride may provide the hydrophilic property of an amphiphilic compound and may include, but is not limited to, PEG 5-1000 or any range therein, and any combination thereof. The glyceride portion of a PEG glyceride may provide the hydrophobic property of an amphiphilic compound and may include, but is not limited to, a natural and/or hydrogenated oil, such as but not limited to, castor oil, hydrogenated castor oil, vitamin A, vitamin D, vitamin E, vitamin K, a plant oil (e.g., corn oil, olive oil, peanut oil, palm kernel oil, apricot kernel oil, almond oil, etc.), and any combination thereof. Exemplary polyethylene glycol (PEG) glycerides include, but are not limited to, PEG-20 castor oil, PEG-20 hydrogenated castor oil, PEG-20 corn glycerides, PEG-20 almond glycerides; PEG-23 trioleate, PEG-40 palm kernel oil, PEG-8 caprylic/capric glycerides, PEG-6 caprylic/capric glycerides, lauroyl macrogol-32 glyceride, stearoyl macrogol glyceride, tocopheryl PEG-1000 succinate, and any combination thereof. In some embodiments of the present disclosure a fatty acid ester comprises a PEG 5-30 (i.e., PEG 5, 6, 7, 8, 9, 10, etc.) and a caprylic/capric glyceride. In some embodiments, a pharmaceutical composition comprises a PEG-5-caprylic/capric glyceride, a PEG-6-caprylic/capric glyceride, a PEG-7-caprylic/capric glyceride, and/or a PEG-8-caprylic/capric glyceride. In certain embodiments, a pharmaceutical composition comprises one or more fatty acid esters such as, but not limited to, those commercially available from Sasol of Hamburg, Germany under the trademark SOFTIGEN®.

An amphiphilic compound may be present in a pharmaceutical composition of the present embodiments at a concentration from about 1% to about 30% by weight or any range therein, such as, but not limited to, from about 2% to about 20% by weight or about 5% to about 15% by weight. In certain embodiments, an amphiphilic compound is present in the pharmaceutical composition at a concentration of about 10% by weight.

A pharmaceutical composition of the present embodiments may further comprise one or more excipients. Excipients for use in pharmaceutical compositions are well-known in the art and examples may be found in the Handbook of Pharmaceutical Excipients (Rowe, R. C. et al., APhA Publications; 5th ed., 2005). Classes of excipients may include, but are not limited to, an emollient, a humectant, a cosolvent, a pH modifier, a water repelling agent, an anti-foaming agent, a surfactant, a solubilizer, a wetting agent, a penetration enhancer, an antioxidant, and/or a solvent. The excipients may be present in a pharmaceutical composition of the present embodiments at any suitable concentration.

In some embodiments, a pharmaceutical composition may further comprise a cosolvent. A cosolvent may be present in a pharmaceutical composition of the present embodiments at a concentration from about 1% to about 30% by weight or any range therein, such as, but not limited to, from about 2% to about 20% by weight or about 5% to about 15% by weight. In certain embodiments, the cosolvent is present in the pharmaceutical composition at a concentration from about 10% to about 15% by weight.

Exemplary cosolvents include, but are not limited to, a fatty acid ester, propylene glycol, glycerol, polyethylene glycol. In some embodiments, a cosolvent may comprise a neutral oil. In certain embodiments, a cosolvent comprises a caprylic and/or capric triglyceride such as, but not limited to, those commercially available from Sasol of Hamburg, Germany under the trademark MIGLYOL®.

The pharmaceutical compositions of the present embodiments may comprise a humectant. Any suitable humectant or combination of humectants may be used. A humectant may be present in a pharmaceutical composition of the present embodiments at a concentration from about 1% to about 25% by weight or any range therein, such as, but not limited to, from about 2% to about 20% by weight or about 5% to about 15% by weight. In certain embodiments, a humectant is present in the pharmaceutical composition at a concentration from about 10% to about 15% by weight.

Exemplary humectants include, but are not limited to, glycols, such as a polyhydric alcohol, diethylene glycol monoethyl ether and methoxypolyethylene glycol; glycerols such as propylene glycol, glycerol, isopropanol, ethanol, ethylene glycol, polyethylene glycol, ethoxydiglycol or mixtures thereof; sugar polyols, such as sorbitol, xylitol and maltitol; polyols such as polydextroses; dimethyl isosorbide; quillaia; urea; and any combination thereof. In some embodiments, a humectant comprises an alkylene glycol, such as hexylene glycol, butylene glycol, pentylene glycol, and any combination thereof.

### Oral Care Compositions

The electrochemical composition may be an oral care composition to be topically applied to the mucosal tissue of the oral cavity, to the gingival tissue of the oral cavity, to the surface of the teeth or any combination thereof. Examples of oral conditions such oral care actives address include, but are not limited to, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores and tooth abscesses, oral malodor, dental erosion, gingivitis, and/or periodontal disease. Oral conditions are further described in WO 02/02096A2.

The electrochemical composition may comprise one or more oral care actives. The oral care active can be any material that is generally recognized as safe for use in the oral cavity that provides changes to the overall health of the oral cavity, and specifically the condition of the oral surfaces that the oral care active contacts.

It is also contemplated that a single oral care product can comprise multiple electrochemical compositions, each of which comprises one or more oral care additives. Some oral care additives that are suitable for use in the electrochemical composition are discussed more fully below.

The electrochemical composition may include one or more gelling agents, which may also act as an adhesive agent to adhere the electrochemical composition to the plurality of teeth. The concentration of the gelling agent may be greater than about 2, 4, 6, 8, 10, 15, 20, 30, 40, 50, 60 percent by weight of the electrochemical composition or less than about 80, 70, 60, 50, 40, 30, or 20 percent by weight of the electrochemical composition.

Suitable gelling agents and/or adhesion agents useful in the present embodiments are described in U.S. Pat. Nos. 6,649,147; 6,780,401; 2004/0102554; 2005/0089819; 2003/0152528; 6,419,906; and 2005/0100515. Some of the gelling agents or adhesion agents may include silicone, polyethylene oxide, polyvinyl alcohol, poly(alkyl vinyl ether-maleic acid) copolymer (PVM/MA copolymer) such as, Gantrez AN 119, AN 139, and S-97, polyvinyl alcohol, polyacrylic acid, Poloxamer 407 (Pluronic), poly(vinylpyrrolidone-vinyl acetate) copolymer (PVP/VA copolymer), such as Luviskol VA, and Plasdone S PVP/VA, polyvinyl pyrrolidone (PVP, e.g., K-15 to K-120), Polyquaternium-11 (Gafquat 755N), Polyquaternium-39 (Merquat plus 3330), carbomer or carboxypolymethylene (Carbopol), hydroxypropyl methylcellulose, hydroxyethyl cellulose, hydroxypropyl cellulose, carboxymethyl cellulose, gelatin, and alginate salt such as sodium alginate, natural gums such as gum karaya, xanthan gum, Guar gum, gum arabic, gum tragacanth, and mixtures thereof.

A humectant or plasticizer may be included in the electrochemical composition, including glycerin, sorbitol, polyethylene glycol, propylene glycol, and other edible polyhydric alcohols. The humectants may be present between about 10% to about 95%, or between about 50% and about 80%, by weight of the electrochemical composition. An electrochemical composition can also include flavoring agents, sweetening agents, opacifiers, and coloring agents.

The electrochemical composition of the present embodiments may comprise a non-electrochemical anti-tartar agent. Anti-tartar actives known for use in dental care products includes phosphates. Phosphates include pyrophosphates, polyphosphates, polyphosphates and mixtures thereof. Pyrophosphates are among the best known for use in dental care products. Pyrophosphate ions are delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms. In one embodiment the electrochemical composition comprises from about 0.5% to about 5% of a pyrophosphate by weight of the electrochemical composition. In another embodiment the electrochemical composition comprises from about 0.5% to about 3% of a pyrophosphate by weight of the electrochemical composition.

The pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated into Kirk & Othmer. Additional anticalculus actives include pyrophosphates or polyphosphates disclosed in U.S. Pat. No. 4,590,066 issued to Parran & Sakkab on May 20, 1986; polyacrylates and other polycarboxylates such as those disclosed in U.S. Pat. No. 3,429,963 issued to Shedlovsky on Feb. 25, 1969 and U.S. Pat. No. 4,304,766 issued to Chang on Dec. 8, 1981; and U.S. Pat. No. 4,661,341 issued to Benedict & Sunberg on Apr. 28, 1987; polyepoxy succinates such as those disclosed in U.S. Pat. No. 4,846,650 issued to Benedict, Bush & Sunberg on Jul. 11, 1989; ethylenediaminetetraacetic acid as disclosed in British Patent No. 490,384 dated Feb. 15, 1937; nitrilotriacetic acid and related compounds as disclosed in U.S. Pat. No. 3,678,154 issued to Widder & Briner on Jul. 18, 1972; polyphosphonates as disclosed in U.S. Pat. No. 3,737,533 issued to Francis on Jun. 5, 1973, U.S. Pat. No. 3,988,443 issued to Ploger, Schmidt-Dunker & Gloxhuber on Oct. 26, 1976 and U.S. Pat. No. 4,877,603 issued to Degenhardt & Kozikowski on Oct. 31, 1989; all of these patents are incorporated herein by reference. Anticalculus phosphates include potassium and sodium pyrophosphates; sodium tripolyphosphate; diphosphonates, such as ethane-1-hydroxy-1,1-diphosphonate, 1-azacycloheptane-1,1-diphosphonate, and linear alkyl diphosphonates; linear carboxylic acids; and sodium zinc citrate.

Actives that may be used in place of or in combination with the pyrophosphate salt include such known materials as synthetic anionic polymers including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Other anti-tartar actives include sodium hexametaphosphate.

The electrochemical composition of the present embodiments may also comprise a non-electrochemical anti-caries agent. Fluoride ion sources are well known for use in oral care compositions as anti-caries actives. Fluoride ions are contained in a number of oral care compositions for this purpose, including without limitation toothpastes. Patents disclosing such toothpastes include U.S. Pat. No. 3,538,230, Nov. 3, 1970 to Pader et al; U.S. Pat. No. 3,689,637, Sep. 5, 1972 to Pader; U.S. Pat. No. 3,711,604, Jan. 16, 1973 to Colodney et al; U.S. Pat. No. 3,911,104, Oct. 7, 1975 to Harrison; U.S. Pat. No. 3,935,306, Jan. 27, 1976 to Roberts et al; and U.S. Pat. No. 4,040,858, Aug. 9, 1977 to Wason.

Application of fluoride ions to dental enamel serves to protect teeth against decay. A wide variety of fluoride ion-yielding materials can be employed as sources of soluble fluoride in the instant electrochemical compositions. Examples of suitable fluoride ion-yielding materials are found in U.S. Pat. Nos. 3,535,421 and 3,678,154. Fluoride ion sources for use herein include stannous fluoride, monofluorophosphate, sodium fluoride, potassium fluoride and ammonium fluoride. In some embodiments, the instant electrochemical compositions provide from about 50 ppm to 10,000 ppm, or from about 100 to 3000 ppm, of fluoride ions in the aqueous solutions that contact dental surfaces when used with the strip of material used in the mouth. Other anti-caries actives include xylitol.

The electrochemical composition of the present disclosure may comprise a non-electrochemical antimicrobial agent. Non-electrochemical antimicrobial agents may include, but are not limited to, 5-chloro-2-(2,4-dichlorophenoxy)-phenol, commonly referred to as triclosan, and described in The Merck Index, 11th ed. (1989), pp. 1529 (entry no. 9573), in U.S. Pat. No. 3,506,720, and in European Patent Application No. 0,251,591; phthalic acid and its salts including, but not limited to those disclosed in U.S. Pat. No. 4,994,262, substituted monoperthalic acid and its salts and esters as disclosed in U.S. Pat. Nos. 4,990,329, 5,110,583, and 4,716,035; magnesium monoperoxyphthalate, chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecyl pyridinium chloride (TPC); N-tetradecyl-4-ethylpyridinium chloride (TDEPC); octenidine; delmopinol, octapinol, and other piperidino derivatives; niacin preparations; zinc/stannous ion actives; antibiotics such as augmentin, amoxicillin, tetracycline, doxycycline, minocycline, and metronidazole; and analogs and salts of the above; essential oils including thymol, geraniol, carvacrol, citral, hinokitiol, eucalyptol, catechol (including 4-allyl catechol), metals or metal ions (e.g., silver, copper, zinc, etc) and mixtures thereof; methyl salicylate; chlorite and metal salts of chlorite and mixtures of all of the above.

The electrochemical composition of the present embodiments may comprise a non-electrochemical anti-inflammatory or non-electrochemical anti-sensitivity agent. Anti-inflammatory agents may include, but are not limited to, non-steroidal anti-inflammatory actives or NSAIDs such as ketorolac, flurbiprofen, ibuprofen, naproxen, indomethacin, aspirin, ketoprofen, piroxicam and meclofenamic acid (and including, but not limited to those disclosed in U.S. Pat. No. 5,626,838). Anti-sensitivity agents can include potassium nitrate, clove oil (Eugenol), and other herbal or flavor actives/agents.

Nutrients may improve the condition of the oral cavity and can be included in the electrochemical compositions. The electrochemical composition of the present embodiments may comprise a non-electrochemical nutrient adjunct include minerals, vitamins, oral nutritional supplements, enteral nutritional supplements, and mixtures thereof.

Minerals that can be included with the electrochemical compositions of the present embodiments include calcium, phosphorus, fluoride, zinc, manganese, potassium and mixtures thereof. These minerals are disclosed in Drug Facts and Comparisons (loose leaf drug information service), Wolters Kluer Company, St. Louis, Mo., COPYRIGHT. 1997, pp 10-17; incorporated herein by reference.

Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Such vitamins are disclosed in Drug Facts and Comparisons (loose leaf drug information service), Wolters Kluer Company, St. Louis, Mo., COPYRIGHT. 1997, pp. 3-10; incorporated herein by reference.

Oral nutritional supplements include amino acids, lipotropics, fish oil, and mixtures thereof, as disclosed in Drug Facts and Comparisons (loose leaf drug information service), Wolters Kluer Company, St. Louis, Mo., COPYRIGHT. 1997, pp. 54-54e; incorporated herein by reference. Amino acids include, but, are not limited to L-Tryptophan, L-Lysine, Methionine, Threonine, Levocamitine or L-carnitine and mixtures thereof. Lipotropics include, but, are not limited to choline, inositol, betaine, linoleic acid, linolenic acid, and mixtures thereof. Fish oil contains large amounts of Omega-3 (N-3) Polyunsaturated fatty acids, eicosapentaenoic acid and docosahexaenoic acid.

Additional nutritional supplements include, but, are not limited to protein products, glucose polymers, corn oil, safflower oil, medium chain triglycerides as disclosed in Drug Facts and Comparisons (loose leaf drug information service), Wolters Kluer Company, St. Louis, Mo., COPYRIGHT. 1997, pp. 55-57; incorporated herein by reference.

### pH Adjustment Agent

Alkaline Material

An alkaline material may be present to trim the pH and/or maintain the pH of the composition according to the present embodiments. The amount of alkaline material is from about 0.001% to about 20%, from about 0.01% to about 10%, or from about 0.05% to about 3% by weight of the composition.

Examples of the alkaline material are sodium hydroxide, potassium hydroxide and/or lithium hydroxide, and/or the alkali metal oxide, such as sodium and/or potassium oxide, or mixtures thereof.

Acidic Material

The electrochemical composition of the present embodiments may comprise an acid. Any acid known to those skilled in the art may be used herein. Typically, the composition herein may comprise up to about 20%, from about 0.1% to about 10%, from about 0.1% to about 5%, from about 0.1% to about 3%, by weight of the total composition of an acid.

Suitable acids are selected from the group consisting of a mono- and poly-carboxylic acid or a mixture thereof a percarboxylic acid or a mixture thereof; a substituted carboxylic acid or a mixture thereof; and mixtures thereof. Carboxylic acids useful herein include Ci-6 linear or at least about 3 carbon containing cyclic acids. The linear or cyclic carbon-containing chain of the carboxylic acid may be substituted with a substituent group selected from the group consisting of hydroxyl, ester, ether, aliphatic groups having from about 1 to about 6, from about 1 to about 4 carbon atoms, and mixtures thereof.

Suitable mono- and poly-carboxylic acids are selected from the group consisting of citric acid, lactic acid, ascorbic acid, isoascorbic acid, tartaric acid, formic acid, maleic acid, malic acid, malonic acid, propionic acid, acetic acid, dehydroacetic acid, benzoic acid, hydroxybenzoic acid, and mixtures thereof.

Suitable percarboxylic acids are selected from the group consisting of peracetic acid, percarbonic acid, perboric acid, and mixtures thereof.

Suitable substituted carboxylic acids are selected from the group consisting of an amino acid or a mixture thereof; a halogenated carboxylic acid or a mixture thereof; and mixtures thereof.

Suitable acids are commercially available from JBL, T&L, or Sigma. Lactic acid is commercially available from Sigma and Purac.

### Methods of Use

The present embodiments further relate to methods of using the compositions to provide disinfecting, and health benefits.

The present embodiments further relate to a method for treating wounds comprising contacting the wound in need of treatment with a composition of the present embodiments and exposing the composition to a current.

The present embodiments further encompass a method of disinfecting a surface, the method comprising the steps of contacting the surface with a composition of the present embodiments and exposing the composition to a current.

The present embodiments further encompass a method of removing biofilm from a surface, the method comprising the steps of contacting the biofilm with a composition of the present embodiments and exposing the composition a current.

The present embodiments further relate to a method for treating or cleaning the oral cavity, including teeth or dentures (inside or outside the oral cavity), comprising contacting the oral cavity (including teeth or dentures) in need of treatment or cleaning with the electrochemical composition, and exposing the composition to a current.

### Packaging

The electrochemical compositions of the present embodiments may be packed in any suitable packaging for delivering the electrochemical compositions for use. In one embodiment, the package may be comprised of polyethylene terephthalate, high-density polyethylene, low-density polyethylene, or combinations thereof. Furthermore, the package may be dosed through a cap at the top of the package such that the composition exits the bottle through an opening in the cap. In one embodiment, the opening in the cap may also contain a screen to help facilitate dosing.

In another embodiment, the package may comprise multiple compartments, two compartments, with a first composition in a first compartment and a second composition in a second compartment. It will be understood that the electrochemical mediator and nitrite salt may be included in either or both of the first and second compartments. In one embodiment, the first composition may comprise the electrochemical mediator and the second composition may comprise the nitrite salt.

In addition to the aspects and embodiments described and provided elsewhere in the present disclosure, the following non-limiting list of embodiments are also contemplated.

1. A therapeutic dressing comprising:

a composition comprising:
an organic electrochemical mediator configured to reduce a gasotransmitter salt; and
the gasotransmitter salt converting into a gasotransmitter upon reduction;

a carrier adapted to contain the composition; and

a bimetallic cell delivering current to the composition.

2. The therapeutic dressing of clause 1, wherein the gasotransmitter salt is selected from the group consisting of nitrate, nitrite, sulfate, thiosulfate, and sulfite salts, and combinations thereof.

3. The therapeutic dressing of clause 1 or clause 2, wherein the gasotransmitter salt is a nitrite salt, and wherein the nitrite salt is selected from the group consisting of nitrite salts of sodium, potassium, calcium, and magnesium, and combinations thereof.

4. The therapeutic dressing of clause 1 or clause 2, wherein the gasotransmitter salt is a sulfite salt, and wherein the sulfite salt is selected from the group consisting of sulfite salts of sodium, potassium, calcium, and magnesium, and combinations thereof.

5. The therapeutic dressing of any one of clauses 1 to 4, wherein the electrochemical mediator has a reduction potential from about −0.1 V to about −2.0 V.

6. The therapeutic dressing of any one of clauses 1 to 5, wherein the electrochemical mediator has a reduction potential from about −0.5 V to about −1.7 V.

7. The therapeutic dressing of any one of clauses 1 to 6, wherein the electrochemical mediator has a reduction potential from about −0.75 V to about −1.5 V.

8. The therapeutic dressing of any one of clauses 1 to 7, wherein the electrochemical mediator is selected from the group consisting of benzophenones, quinones, and derivatives thereof, and combinations thereof.

9. The therapeutic dressing of any one of clauses 1 to 8, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

10. The therapeutic dressing of any one of clauses 1 to 7, wherein the electrochemical mediator is selected from the group consisting of fluorescein, xanthone, thioxanthone, and derivatives thereof, and combinations thereof.

11. The therapeutic dressing of clause 1 or clause 10, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

12. The therapeutic dressing of clause 1, wherein the gasotransmitter salt comprises a nitrite salt or nitrate salt which converts into a nitric oxide via electron transfer.

13. The therapeutic dressing of clause 1 or clause 12, wherein the gasotransmitter salt comprises the nitrite salt having the formula:

A[NO2]ₘ

wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium cations, and combinations thereof.

14. The therapeutic dressing of clause 1 or clause 12, wherein the nitrate salt or nitrite salt has a cation consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc cations, and combinations thereof.

15. The therapeutic dressing of clause 1, wherein the gasotransmitter salt comprises a sulfite salt, sulfate salt, or thiosulfate salt which converts into a hydrogen sulfide via electron transfer.

16. The therapeutic dressing of clause 1 or clause 15, wherein the gasotransmitter salt comprises the sulfite salt having the formula:

Aₙ[SO₃]ₘ

wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium cations, and combinations thereof.

17. The therapeutic dressing of clause 1 or clause 15, wherein the sulfite salt, sulfate salt, or thiosulfate salt has a cation selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc cations, and combinations thereof.

18. The therapeutic dressing of clause 1, wherein the electrochemical mediator is a water soluble ketone or derivative thereof.

19. The therapeutic dressing of clause 1 or clause 18, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, ethylene imine, and combinations thereof.

20. The therapeutic dressing of clause 1, wherein the bimetallic cell further comprises an anode, wherein the anode is selected from the group consisting of magnesium, zinc, aluminum, and copper, and combinations thereof.

21. The therapeutic dressing of clause 20, wherein the anode is zinc or aluminum.

22. The therapeutic dressing of clause 1, wherein the bimetallic cell further comprises a cathode, wherein the cathode is selected from the group consisting of carbon, silver, gold, and platinum, and combinations thereof.

23. The therapeutic dressing of clause 22, wherein the cathode is carbon or silver.

24. The therapeutic dressing of clause 1, wherein the bimetallic cell further comprises a cathode, wherein the cathode is graphite or carbon.

25. A process for the preparation of a gasotransmitter, the process comprising exposing the composition of any one of clauses 1 to 24 to a reduction potential from about −0.1 V to about −2.0 V.

26. A method of treating a wound, the method comprising:
contacting the wound with the composition of any one of clauses 1 to 24; and
exposing the composition to a voltage from a bimetallic cell.

27. A method of treating acne vulgaris, the method comprising:
contacting the acne vulgaris with the composition of any one of clauses 1 to 24; and
exposing the composition to a voltage from a bimetallic cell.

28. A method of treating skin ulcers, the method comprising:
contacting the skin ulcers with the composition of any one of clauses 1 to 24; and
exposing the composition to a voltage from a bimetallic cell.

29. A method of treating a virus, the method comprising:
contacting the virus with the composition of any one of clauses 1 to 24; and
exposing the composition to a voltage from a bimetallic cell.

30. A method of removing biofilm from a surface, the method comprising:
contacting the biofilm with the composition of any one of clauses 1 to 24; and
exposing the composition to a voltage from a bimetallic cell.

31. A composition comprising:
an organic electrochemical mediator; and
a gasotransmitter salt which converts into a gasotransmitter upon reduction;
wherein the composition is in contact with an anode and a cathode selected from a galvanic series.

32. The composition of clause 31, wherein the gasotransmitter salt is selected from the group consisting of nitrate, nitrite, sulfate, thiosulfate, and sulfite salts.

33. The composition of clause 31 or clause 32, wherein the electrochemical mediator has a reduction potential from about −0.1 V to about −2.0 V.

34. The composition of any one of clauses 31 to 33, wherein the electrochemical mediator has a reduction potential from about −0.5 V to about −1.7 V.

35. The composition of any one of clauses 31 to 34, wherein the electrochemical mediator has a reduction potential from about −0.75 V to about −1.5 V.

36. The composition of any one of clauses 31 to 35, wherein the electrochemical mediator is selected from the group consisting of water soluble ketones, benzophenones, and quinones.

37. The composition of any one of clauses 31 to 35, wherein the electrochemical mediator is selected from the group consisting of fluorescein, xanthone and thioxanthone.

38. The composition of any one of clauses 31 to 35, wherein:
the organic electrochemical mediator is a water soluble organic electrochemical mediator having a redox moiety and a water solubilizing moiety; and
the gasotransmitter salt is a nitrite or nitrate salt which converts into a nitric oxide via electron transfer.

39. The composition of any one of clauses 31 to 35, wherein the composition comprises:
the organic electrochemical mediator is a water soluble organic electrochemical mediator having a redox moiety and a water solubilizing moiety; and
the gasotransmitter salt is a sulfite, sulfate or thiosulfate salt which converts into a hydrogen sulfide via electron transfer.

40. The composition of clause 31 or clause 38, wherein the gasotransmitter salt is a nitrite salt having the formula:

A[NO2]ₘ
wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium, and combinations thereof.

41. The composition of clause 31, wherein the cation A is selected from the group consisting of monovalent cations, divalent cations, and trivalent cations selected from the group consisting of lithium, sodium, potassium, magnesium, calcium, and ammonium cations, and combinations thereof.

42. The composition of clause 31 or clause 39, wherein the gasotransmitter salt is a sulfite salt having the formula:

Aₙ[SO₃]ₘ
wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium, and combinations thereof.

43. The composition of clause 42, wherein the cation A is selected from the group consisting of monovalent cations, divalent cations, and trivalent cations selected from the group consisting of lithium, sodium, potassium, magnesium, calcium, and ammonium cations, and combinations thereof.

44. The composition of clause 38, wherein the nitrate or nitrite salt has a cation selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc, and combinations thereof.

45. The composition of clause 39, wherein the sulfate, thiosulfate or sulfite salt has a cation selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc cations, and combinations thereof.

46. The composition of clause 37, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

47. The composition of clause 38, wherein the redox moiety is selected from the group consisting of ketone moieties, benzophenone moieties, and quinone moieties, and combinations thereof.

48. A therapeutic dressing comprising:
a composition of any one of clauses 31 to 47;
a carrier adapted to contain the composition; and
two dissimilar electrodes selected from the galvanic series in electrical contact with the composition.

49. A bandage comprising a dressing of clause 48.

50. A bandage comprising:
a dressing comprising:
   an organic electrochemical mediator; and
   a gasotransmitter salt which converts into a gasotransmitter; and
an anode and cathode selected from the galvanic series and positioned to be capable of applying a current to the dressing, whereby to convert the gasotransmitter salt into a gasotransmitter.

51. The bandage of clause 50, wherein at least one of the anode and cathode is moveable from a first position in electrical contact with the dressing to a second position not in electrical contact with the dressing.

52. The bandage of clause 50, further comprising at least one removable insulator or spacer material disposed between at least one of the anode and cathode and the dressing whereby, upon its removal to bring the at least one of the anode and cathode in electrical contact with the dressing.

### Indigo Carmine Bleaching Test Method

The generation of the gasotransmitters generated from the present embodiments can be evaluated using an indigo carmine bleaching method.

A solution of the electrochemical mediator is prepared in 1% aqueous gasotransmitter salt solution containing 2 ppm Indigo carmine as a bleaching indicator. A UV/Vis spectra is recorded. The solution is placed in a cell comprising two 2 cm by 2 cm electrodes selected from a galvanic series. A UV/Vis spectra is recorded after ten minutes. The reduction in the intensity of the Indigo carmine visible absorption peak at ˜610 nm is used to determine the efficacy of the activator. Activators of the present disclosure are considered suitable if the Indigo carmine absorption peak intensity was reduced by more than a control solution that does not contain a gasotransmitter salt.

### Solution Nitric Oxide Test Method

Nitric oxide generated from the present embodiments can be evaluated using the following method.

A solution of the electrochemical mediator is prepared containing 1% of a nitrite or nitrate salt, as described above, and 10 ppm 1,2-diaminoanthraquinone in 75/25 v/v water/isopropyl alcohol. A UV/Vis spectra is recorded.

The solution is placed in a cell comprising electrodes selected from the galvanic series as previously described. A UV/Vis spectra is recorded after ten minutes. The reduction in the intensity of the 1,2-diamino anthraquinone visible absorption peak at 540 nm is used to determine the efficacy of the activator. Activators and bimetallic electrodes of the present embodiments are considered suitable if the absorption peak intensity is reduced by more than a control.

### Gel Nitric Oxide Test Method

DAQ Gelatin

The transfer of nitric oxide from a formulation across an interface into a substrate is demonstrated using a gel containing 1,2-diaminoanthraquinone. Gelatin (10% w/v) is dissolved in water at approximately 90° C. The hot gelatin solution is divided into 50 ml portions and poured into 14 cm diameter petri dishes and 5 ml of 1,2-diaminoanthraquinone (400 ppm w/v in isopropyl alcohol) is added to the hot gelatin in the petri dish and dispersed by stirring. The gelatin is allowed to cool to below 30 C to form ‘DAQ gelatin.’

Super Absorbing Hydrogel Dressing

Gauze dressing material is sealed on 3 sides to form a pouch. 2 g of crosslinked sodium polyacrylate (9003-04-7, Sigma Aldrich) is placed in the gauze pouch and the fourth side is sealed.

Bimetallic Cell Hydrogel Dressing

Gauze dressing material is sealed on 3 sides to form a pouch. 2 g of crosslinked sodium polyacrylate (9003-04-7, Sigma Aldrich) is placed in the gauze pouch along with electrodes selected from the galvanic series. The fourth side of the dressing is sealed.

Bimetallic Cell Dressing

Electrodes selected from the galvanic series are attached to the surface of a commercial adhesive dressing.

### EXAMPLES

### Example 1—Nitric Oxide Generating Compositions

Topical nitric oxide generating compositions are prepared according to Table 1.

TABLE 1

| Ingredient % w/w | A | B | C | D |
|---|---|---|---|---|
| Purified Water, USP | 85.0 | 85.0 | 85.0 | 85.0 |
| Sodium Nitrite | 1.0 | 3.0 | 0.5 | 5.0 |
| Benzophenone | 0.01 | 0.1 | 0.001 | 0.5 |
| tetracarboxylic acid | | | | |
| Glycerol, NF | 10.0 | 10.0 | 10.0 | 10.0 |
| Diaminoanthroquinone | 0.01 | 0.01 | 0.01 | 0.01 |
| Carbomer Homopolymer | 1.0 | 0.3 | — | |
| Type A, NF Carbopol 974P | | | | |
| Carbomer Interpolymer | — | — | 0.5 | 1.0 |
| Type B, NF Carbopol | | | | |
| ETD2020NF | | | | |
| Trolamine, NF | To pH 7 | — | | To pH 7 |
| 0.1M Phosphate Buffer | — | To pH 7 | To pH 7 | — |
| Purified water USP | To 100 | To 100 | To 100 | To 100 |

The formulations are placed in a cell comprising electrodes selected from the bimetallic series as described above. A UV/Vis spectra is recorded after ten minutes. The reduction in the intensity of the 1,2-diamino anthraquinone visible absorption peak at 540 nm indicates that nitric oxide is formed.

### Example 2—Gasotransmitter Formulation

Aqueous formulations are prepared according to Table 2.

TABLE 2

| Ingredient | A | B | C | D | E | F |
|---|---|---|---|---|---|---|
| Sodium Nitrite | 0.5 | 1 | 2 | — | — | — |
| (% w/v) | | | | | | |
| Sodium Sulfite | — | — | — | 0.5 | 1 | 2 |
| (% w/v) | | | | | | |
| Benzophenone | 100 | 50 | 100 | 100 | 50 | 100 |
| tetracarboxylic | | | | | | |
| acid (ppm) | | | | | | |
| 0.1M Phosphate | To | To | To | To | To | To |
| Buffer | pH 7 | pH 7 | pH 7 | pH 7 | pH 7 | pH 7 |
| Purified water | To | To | To | To | To | To |
| USP | 100 | 100 | 100 | 100 | 100 | 100 |

### Example 3—Bimetallic Cell Hydrogel Dressing Evaluation 1

A bimetallic cell hydrogel dressing containing a carbon and an aluminum electrode was placed in 50 mL of formulation A. The formulation was absorbed into the dressing (2-3 minutes) and the dressing was placed on a DAQ gelatin petri dish. The loss in the red color of the DAQ indicated the generation of nitric oxide and its transfer across the dressing/substrate.

### Example 4—Bimetallic Cell Hydrogel Dressing Evaluation 2

A bimetallic cell hydrogel dressing containing a carbon and a zinc electrode was placed in 50 mL of formulation C. The formulation was absorbed into the dressing (2-3 minutes) and the dressing was placed on a DAQ gelatin petri dish. The loss in the red color of the DAQ indicated the generation of nitric oxide and its transfer across the dressing/substrate.

### Example 5—Bimetallic Cell Hydrogel Dressing Evaluation 3

A bimetallic cell hydrogel dressing containing a carbon and an aluminum electrode was placed in 50 mL of formulation B. The formulation was absorbed into the dressing (2-3 minutes) and the dressing was placed on a DAQ gelatin petri dish. The loss in the red color of the DAQ indicated the generation of nitric oxide and its transfer across the dressing/substrate.

### Example 6—Bimetallic Cell Dressing Evaluation 1

A super absorbent hydrogel dressing was placed in 50 mL of formulation B. The formulation was absorbed into the dressing (2-3 minutes) and the dressing was placed on a DAQ gelatin petri dish. A bimetallic cell dressing with a carbon and an aluminum electrode was placed on top of the hydrogel dressing. The loss in the red color of the DAQ indicated the generation of nitric oxide and its transfer across the dressing/substrate.

### Example 7—Bimetallic Cell Dressing Evaluation 2

A super absorbent hydrogel dressing as described in was placed in 50 mL of formulation A. The formulation was absorbed into the dressing (2-3 minutes) and the dressing was placed on a DAQ gelatin petri dish. A bimetallic cell dressing with a carbon and a zinc electrode was placed on top of the hydrogel dressing. The loss in the red color of the DAQ indicated the generation of nitric oxide and its transfer across the dressing/substrate.

Hydrogen sulfide generated from the present embodiments can be evaluated using the following method.

### Hydrogen Sulfide Test Method

A solution of the electrochemical mediator is prepared containing 1% of a sulfite, sulfate or thiosulfate salt, as described above.

The solution is placed in a cell comprising electrodes selected from the bimetallic series as described above. After 10 minutes of electrolysis the solution is tested for hydrogen sulfide using lead acetate paper, for example. Activators of the present embodiments are considered suitable if the test for hydrogen sulfide is positive

### Example 8—Hydrogen Sulfide Generating Compositions

Topical hydrogen sulfide generating compositions are prepared according to Table 3.

TABLE 3

| Ingredient % w/w | A | B | C | D |
|---|---|---|---|---|
| Purified Water, USP | 85.0 | 85.0 | 85.0 | 85.0 |
| Sodium Sulfite | 1.0 | 3.0 | 0.5 | 5.0 |
| Benzophenone | 0.01 | 0.1 | 0.001 | 0.5 |
| Tetracarboxylic Acid | | | | |
| Glycerol, NF | 10.0 | 10.0 | 10.0 | 10.0 |
| Carbomer Homopolymer | 1.0 | 0.3 | — | — |
| Type A, NF Carbopol 974P | | | | |
| Carbomer Interpolymer | — | — | 0.5 | 1.0 |
| Type B, NF Carbopol | | | | |
| ETD2020NF | | | | |
| Trolamine, NF | To pH 7 | — | — | To pH 7 |
| 0.1M Phosphate Buffer | — | To pH 7 | To pH 7 | — |
| Purified Water USP | To 100 | To 100 | To 100 | To 100 |

The formulations are placed in a cell comprising electrodes selected from the bimetallic series as described above and the generation of hydrogen sulfide was shown using lead acetate paper.

### Example 9—Bimetallic Cell Hydrogel Dressing Evaluation 1

A bimetallic cell hydrogel dressing containing a carbon and an aluminum electrode was placed in 50 mL of formulation D. The formulation was absorbed into the dressing (2-3 minutes). The generation of hydrogen sulfide at the surface of the dressing is confirmed using lead acetate paper.

### Example 10—Bimetallic Cell Dressing Evaluation 1

A super absorbent hydrogel dressing was placed in 50 mL of formulation E. The formulation was absorbed into the dressing (2-3 minutes). A bimetallic cell dressing with a carbon and a zinc electrode was placed on top of the hydrogel dressing. The generation of hydrogen sulfide at the surface of the dressing is confirmed using lead acetate paper.

### Example 11—Carbon/Aluminum Galvanic Dressing Producing Nitric Oxide

A piece of gauze dressing was folded in half and two of the open sides sealed with heat activated adhesive. In the resulting dressing is placed 2 g of crosslinked sodium polyacrylate (supersorber), a 5 cm×2 cm piece of carbon cloth, and a 5 cm×2 cm piece of aluminum foil. The remaining open side is sealed using heat activated adhesive. The resulting dressing was placed in 35 mL of a gasotransmitter solution comprising 2% sodium nitrite and 100 ppm of benzophenone tetracarboxylic acid. The dressing generated nitric oxide.

### Example 12—Carbon/Aluminum Galvanic Dressing Producing Hydrogen Sulfide

A piece of gauze dressing was folded in half and two of the open sides sealed with heat activated adhesive. In the resulting dressing is placed 2 g of crosslinked sodium polyacrylate (supersorber), a 5 cm×2 cm piece of carbon cloth, and a 5 cm×2 cm piece of aluminum foil. The remaining open side is sealed using heat activated adhesive. The resulting dressing was placed in 35 mL of a gasotransmitter solution comprising 2% sodium sulfite and 100 ppm of benzophenone tetracarboxylic acid. The dressing generated hydrogen sulfide.

### Example 13—Carbon/Zinc Galvanic Dressing Producing Nitric Oxide

A piece of gauze dressing was folded in half and two of the open sides sealed with heat activated adhesive. In the resulting dressing is placed 2 g of crosslinked sodium polyacrylate (supersorber), a 5 cm×2 cm piece of carbon cloth, and a 5 cm×2 cm piece of zinc foil. The remaining open side is sealed using heat activated adhesive. The resulting dressing was placed in 35 mL of a gasotransmitter solution comprising 2% sodium nitrite and 100 ppm of benzophenone tetracarboxylic acid. The dressing generated nitric oxide.

### Example 14—Carbon/Zinc Galvanic Dressing Producing Hydrogen Sulfide

A piece of gauze dressing was folded in half and two of the open sides sealed with heat activated adhesive. In the resulting dressing is placed 2 g of crosslinked sodium polyacrylate (supersorber), a 5 cm×2 cm piece of carbon cloth, and a 5 cm×2 cm piece of zinc foil. The remaining open side is sealed using heat activated adhesive. The resulting dressing was placed in 35 mL of a gasotransmitter solution comprising 2% sodium sulfite and 100 ppm of benzophenone tetracarboxylic acid. The dressing generated hydrogen sulfide.

It should be understood that every maximum numerical limitation given throughout this specification would include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

What is claimed is:

1. A therapeutic dressing comprising:
a composition comprising:
an organic electrochemical mediator configured to reduce a gasotransmitter salt; and
the gasotransmitter salt converting into a gasotransmitter upon reduction;
a carrier adapted to contain the composition; and
a bimetallic cell delivering current to the composition.

2. The therapeutic dressing of claim 1, wherein the gasotransmitter salt is selected from the group consisting of nitrate, nitrite, sulfate, thiosulfate, and sulfite salts, and combinations thereof.

3. The therapeutic dressing of claim 2, wherein the gasotransmitter salt is a nitrite salt, and wherein the nitrite salt is selected from the group consisting of nitrite salts of sodium, potassium, calcium, and magnesium, and combinations thereof.

4. The therapeutic dressing of claim 2, wherein the gasotransmitter salt is a sulfite salt, and wherein the sulfite salt is selected from the group consisting of sulfite salts of sodium, potassium, calcium, and magnesium, and combinations thereof.

5. The therapeutic dressing of claim 1, wherein the electrochemical mediator has a reduction potential from about −0.1 V to about −2.0 V.

6. The therapeutic dressing of claim 1, wherein the electrochemical mediator has a reduction potential from about −0.5 V to about −1.7 V.

7. The therapeutic dressing of claim 1, wherein the electrochemical mediator has a reduction potential from about −0.75 V to about −1.5 V.

8. The therapeutic dressing of claim 1, wherein the electrochemical mediator is selected from the group consisting of benzophenones, quinones, and derivatives thereof, and combinations thereof.

9. The therapeutic dressing of claim 8, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

10. The therapeutic dressing of claim 1, wherein the electrochemical mediator is selected from the group consisting of fluorescein, xanthone, thioxanthone, and derivatives thereof, and combinations thereof.

11. The therapeutic dressing of claim 10, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

12. The therapeutic dressing of claim 1, wherein the gasotransmitter salt comprises a nitrite salt or nitrate salt which converts into a nitric oxide via electron transfer.

13. The therapeutic dressing of claim 12, wherein the gasotransmitter salt comprises the nitrite salt having the formula:
A[NO2]ₘ
wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium cations, and combinations thereof.

14. The therapeutic dressing of claim 12, wherein the nitrate salt or nitrite salt has a cation consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc cations, and combinations thereof.

15. The therapeutic dressing of claim 1, wherein the gasotransmitter salt comprises a sulfite salt, sulfate salt, or thiosulfate salt which converts into a hydrogen sulfide via electron transfer.

16. The therapeutic dressing of claim 15, wherein the gasotransmitter salt comprises the sulfite salt having the formula:
Aₙ[SO₃]ₘ
wherein A is selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, zinc, ammonium, alkyl-ammonium, and aryl-ammonium cations, and combinations thereof.

17. The therapeutic dressing of claim 15, wherein the sulfite salt, sulfate salt, or thiosulfate salt has a cation selected from the group consisting of monovalent cations, divalent cations, and trivalent cations selected from the group consisting of aluminum, barium, calcium, cobalt, chromium, copper, iron, lithium, potassium, rubidium, magnesium, manganese, molybdenum, nickel, sodium, titanium, vanadium, and zinc cations, and combinations thereof.

18. The therapeutic dressing of claim 1, wherein the electrochemical mediator is a water soluble ketone or derivative thereof.

19. The therapeutic dressing of claim 18, wherein the electrochemical mediator comprises a redox moiety and a hydrophilic moiety selected from the group consisting of alcohol, amine, amide, carboxylic acid, sulfonic acid, phosphatealkylene oxide oligomers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphonic acid, and ethylene imine, and combinations thereof.

20. The therapeutic dressing of claim 1, wherein the bimetallic cell further comprises an anode, wherein the anode is selected from the group consisting of magnesium, zinc, aluminum, and copper, and combinations thereof.

21. The therapeutic dressing of claim 20, wherein the anode is zinc or aluminum.

22. The therapeutic dressing of claim 1, wherein the bimetallic cell further comprises a cathode, wherein the cathode is selected from the group consisting of carbon, silver, gold, and platinum, and combinations thereof.

23. The therapeutic dressing of claim 22, wherein the cathode is carbon or silver.

24. The therapeutic dressing of claim 1, wherein the bimetallic cell further comprises a cathode, wherein the cathode is graphite.

25. A process for the preparation of a gasotransmitter, the process comprising exposing the composition of claim 1 to a reduction potential from about −0.1 V to about −2.0 V.

26. A method of treating a wound, the method comprising:
contacting the wound with the composition of claim 1; and
exposing the composition to a voltage from a bimetallic cell.

27. A method of treating acne vulgaris, the method comprising:
contacting the acne vulgaris with the composition of claim 1; and
exposing the composition to a voltage from a bimetallic cell.

28. A method of treating skin ulcers, the method comprising:
contacting the skin ulcers with the composition of claim 1; and
exposing the composition to a voltage from a bimetallic cell.

29. A method of treating a virus, the method comprising:
contacting the virus with the composition of claim 1; and
exposing the composition to a voltage from a bimetallic cell.

30. A method of removing biofilm from a surface, the method comprising:
contacting the biofilm with the composition of claim 1; and
exposing the composition to a voltage from a bimetallic cell.
